(19) United States
(12) Patent Application Publication
(10) Pub. No.: US 2020/0046654 A1
(43) Pub. Date: Feb. 13, 2020

(54) BUPROPION AND PHARMACEUTICAL COMPOSITION FOR TREATING CANCER AND METHOD FOR INHIBITING MIGRATION OF TUMOR CELLS

(71) Applicant: NATIONAL CHIAO TUNG UNIVERSITY, Hsinchu City (TW)

(72) Inventors: Jinn-Moon YANG, Hsinchu City (TW); Chun-Yu LIN, Kaohsiung City (TW); Yi-Yuan CHIU, Tainan City (TW); Jung-Yu LEE, Taipei City (TW); Yuan-Soon HO, New Taipei City (TW); Chia-Hwa LEE, Taipei City (TW)

(21) Appl. No.: 16/267,682

(22) Filed: Feb. 5, 2019

(30) Foreign Application Priority Data
Aug. 9, 2018 (TW) 107127866

(51) Int. Cl. A61K 31/135; A61K 9/00; A61P 35/04

(52) U.S. Cl. CPC A61K 31/135; A61P 35/04; A61K 9/0053

(57) ABSTRACT

A method for treating cancer in a subject is provided; the method includes administrating bupropion to the subject, wherein the tumor cells of the cancer overexpress neuronal acetylcholine receptor subunit α9 (CHRNA9). Another method for treating cancer in a subject is provided; the method includes administrating a pharmaceutical composition to the subject, wherein the pharmaceutical composition includes: a therapeutically effective amount of bupropion and a pharmaceutically acceptable excipient; wherein the tumor cells of the cancer overexpress CHRNA9. A method for inhibiting migration of tumor cells is also provided; the method includes administrating an effective amount of bupropion to the tumor cells; wherein the tumor cells overexpress CHRNA9.

### CROSS-REFERENCE TO RELATED APPLICATION

This application claims priority to Taiwan Application Serial Number 107127866, filed Aug. 9, 2018, which is herein incorporated by reference in its entirety.

### BACKGROUND

### Field of Invention

The present disclosure relates to using bupropion as a therapeutic agent for diseases.

### Description of Related Art

Targeted cancer therapy works by targeting specific genes or proteins to help stop cancer from growing and spreading. Unlike chemotherapy, targeted therapy has less effect on normal cells, and the patients' side effects and pain are less severe. However, suitable target drugs for many cancer types have not yet been developed.

Previous studies have shown that expressions of neuronal acetylcholine receptors (nAChRs), e.g., neuronal acetylcholine receptor subunit α7 (α7-nAChR or also called CHRNA7) or neuronal acetylcholine receptor subunit α9 (α9-nAChR or also called CHRNA9), play decisive roles in smoking-induced cancer formation. Therefore, the nAChRs detected in tumor cells may be used as targets for clinical treatment.

Bupropion is a known antidepressant and can be used to treat other conditions such as nicotine addiction, obesity, Parkinson's disease, seasonal affective disorder, etc. Previous studies have shown that bupropion can be an antagonist of some nAChRs. However, the effects of bupropion on the cells expressing these neuronal nAChRs are still unclear.

### SUMMARY

In view of the problem mentioned above, the art is in great need of a drug that can act on a nAChR. The drug can be used for developing targeted therapies to treat cancers with overexpression of the nAChR.

Some embodiments of the present disclosure provide a use of bupropion for the manufacture of a medicament for the treatment of cancer, wherein the tumor cells of the cancer overexpress CHRNA9.

Some embodiments of the present disclosure provide a method for treating cancer in a subject. The method includes: administrating bupropion to the subject, wherein the tumor cells of the cancer overexpress CHRNA9.

Some embodiments of the present disclosure also provide a use of a pharmaceutical composition for the manufacture of a medicament for the treatment of cancer, wherein the pharmaceutical composition includes a therapeutically effective amount of bupropion and a pharmaceutically acceptable excipient, and the tumor cells of the cancer overexpress CHRNA9.

Some embodiments of the present disclosure provide a method for treating cancer in a subject. The method includes administrating a pharmaceutical composition to the subject, wherein the pharmaceutical composition includes a therapeutically effective amount of bupropion and a pharmaceutically acceptable excipient; wherein the tumor cells of the cancer overexpress CHRNA9.

Some embodiments of the present disclosure also provide a method of inhibiting migration of tumor cells overexpressing CHRNA9. The method includes administrating an effective amount of bupropion to the tumor cells.

It is to be understood that both the foregoing general description and the following detailed description are by examples, and are intended to provide further explanation of the invention as claimed.

### BRIEF DESCRIPTION OF THE DRAWINGS

The invention can be more fully understood by reading the following detailed description of the embodiment, with reference made to the accompanying drawings as follows:

FIG. 1A shows a Western Blot image, according to Example 1 of the present disclosure.

FIG. 1B shows a Western Blot image of the immunoprecipitation (IP) samples, according to Example 1 of the present disclosure.

FIG. 10 shows the fluorescence images of the cells, according to Example 1 of the present disclosure.

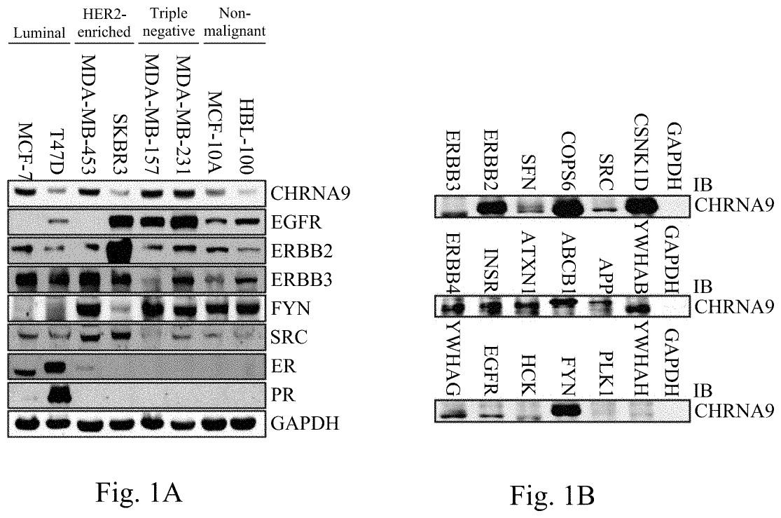

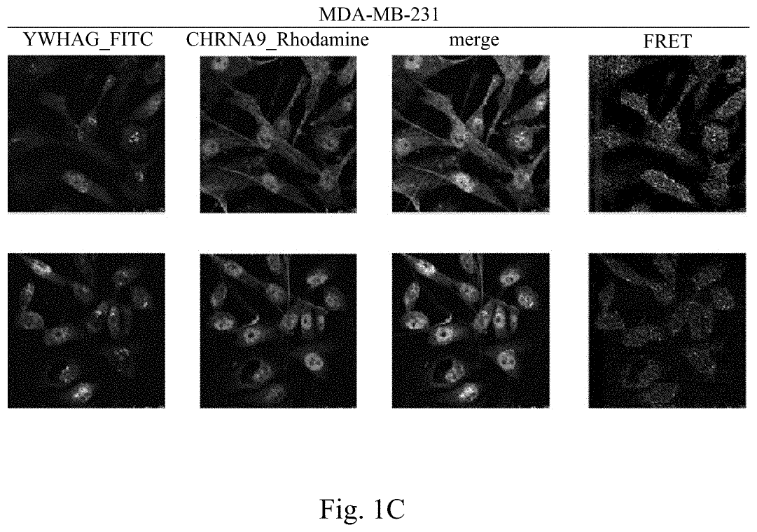

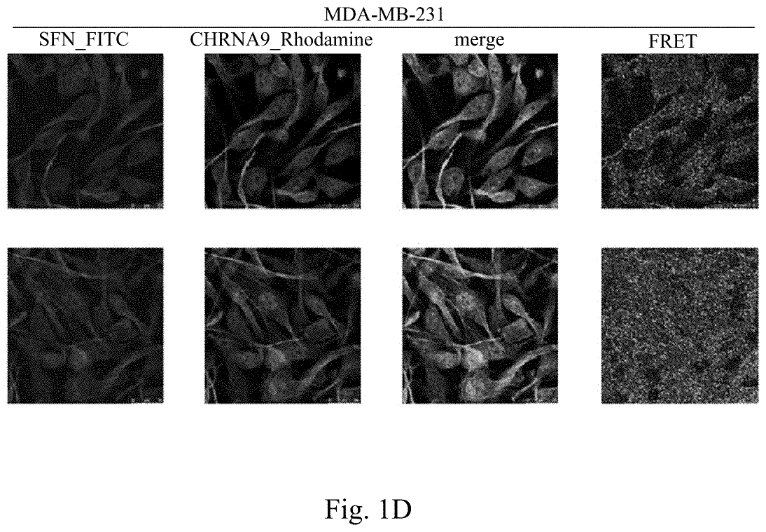

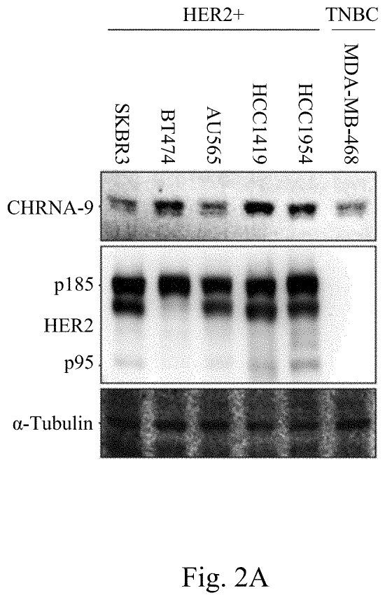

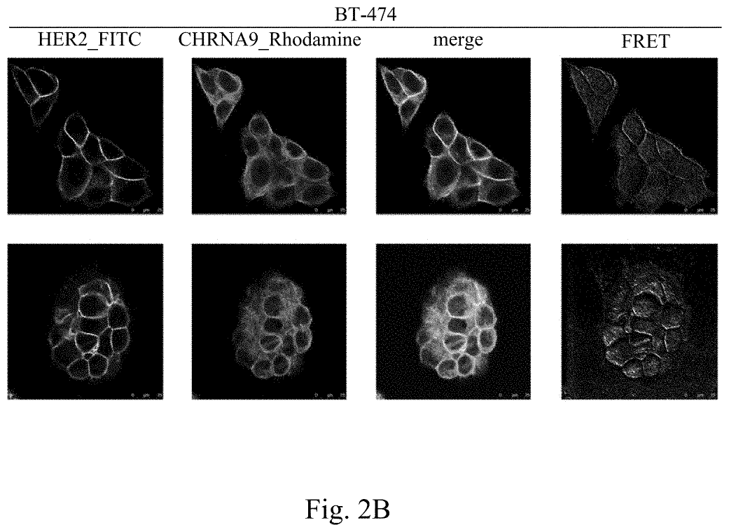

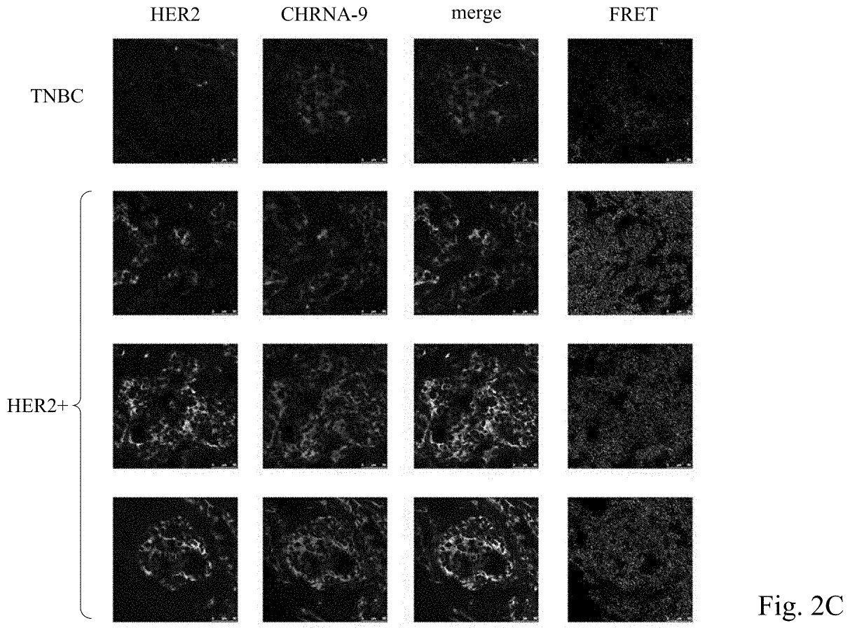

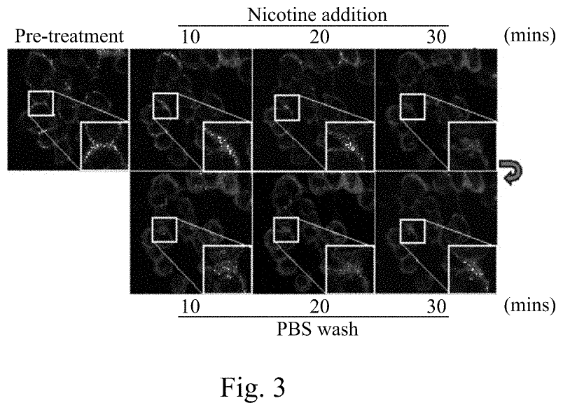

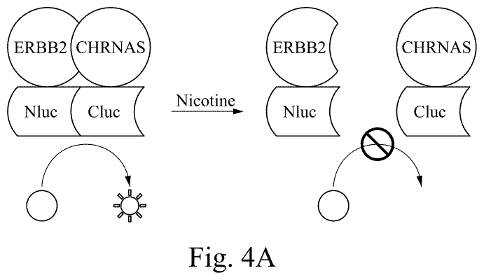

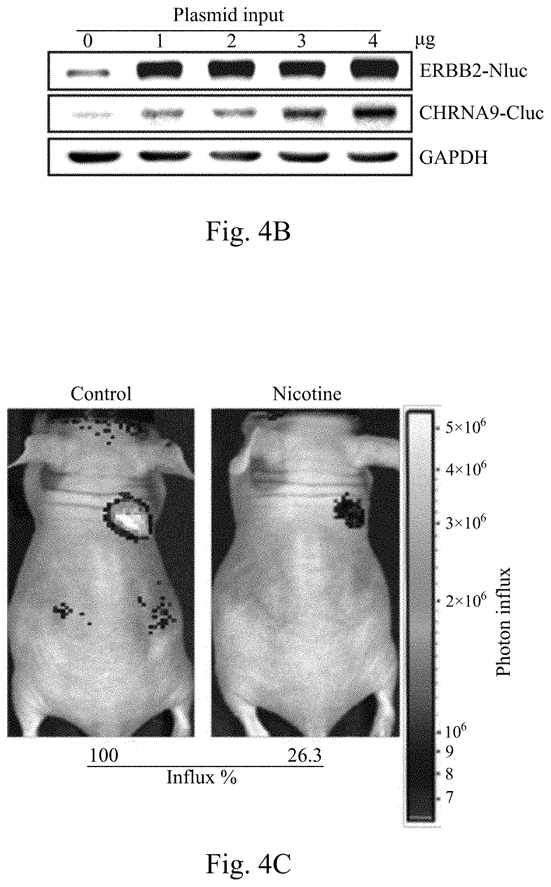

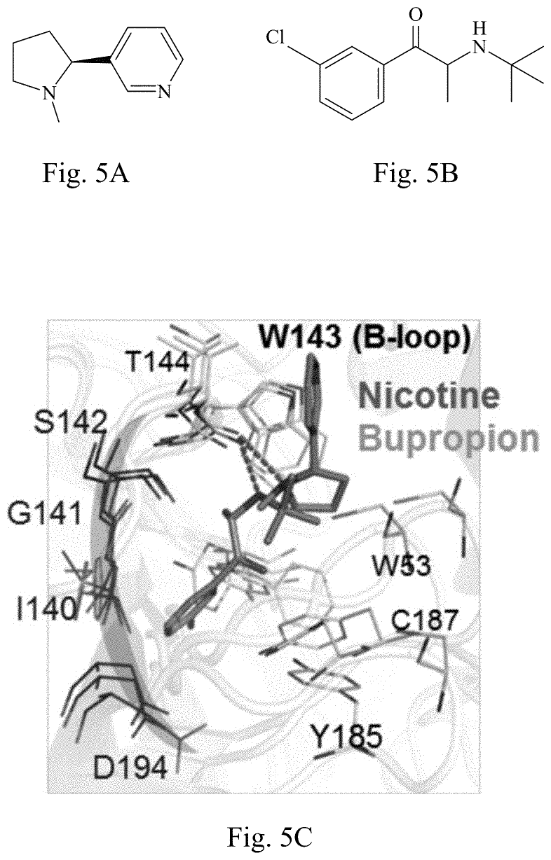

FIG. 1D shows the fluorescence images of the cells, according to Example 1 of the present disclosure.

FIG. 2A shows a Western Blot image, according to Example 2 of the present disclosure.

FIG. 2B shows the fluorescence images of the cells, according to Example 2 of the present disclosure.

FIG. 2C shows the fluorescence images of the cells, according to Example 2 of the present disclosure.

FIG. 3 shows the fluorescence images of the cells, according to Example 3 of the present disclosure.

FIG. 4A shows a schematic diagram of the split luciferase complementation assay, according to Example 4 of the present disclosure.

FIG. 4B shows a Western Blot image, according to Example 4 of the present disclosure.

FIG. 4C is the images taken by a non-invasive in vivo imaging system (IVIS) to show the in vivo luminescent signals in the mice, according to Example 4 of the present disclosure.

FIG. 5A illustrates the molecular structure of nicotine.

FIG. 5B shows the molecular structure of bupropion.

FIG. 5C shows a partial schematic diagram of a protein structure to illustrate the relative spatial locations of bupropion, nicotine and the nearby amino acid residues of CHRNA9.

FIG. 6A shows the images of the chamber surfaces of the cell invasion assay, according to Example 6 of the present disclosure.

FIG. 6B shows the bar graph based on the quantification of the cells in the assay of FIG. 6A, according to Example 6 of the present disclosure.

FIG. 7A shows the images of the cells of the cell migration assay, according to Example 7 of the present disclosure.

FIG. 7B shows the bar graph based on the quantification of the cells in the assay of FIG. 7A, according to Example 7 of the present disclosure.

FIG. 7C shows the cell images of the cell migration assay, according to Example 8 of the present disclosure.

FIG. 7D shows the bar graph based on the quantification of the cells in the assay of FIG. 7C, according to Example 8 of the present disclosure.

FIG. 8A is the luminescence images taken by IVIS to show the photon flux of the lungs from the sacrificed mice, according to Example 9 of the present disclosure.

FIG. 8B shows the bar graph based on the quantification of the photon flux in the experiment of FIG. 8A, according to Example 9 of the present disclosure.

### DETAILED DESCRIPTION

The present disclosure provides many different embodiments, or examples, for implementing different features of this disclosure. Specific examples of components and arrangements are described below to simplify the present disclosure. These are, of course, merely examples and are not intended to be limiting.

As used herein, the term “treating” means reducing the frequency, severity, or duration of cancer symptoms for a subject. The term “treatment” includes the act of reducing or slowing down the severity or symptoms associated with a specific disease or syndrome in one suffering from the disease or syndrome.

As used herein, the terms “subject” and “patient” may be used interchangeably, and the terms refer to an animal, including a human being, can be treated with the compound or pharmaceutical composition disclosed in the present disclosure.

As used herein, the term “effective amount” or “therapeutically effective amount” is a dose of an active ingredient sufficient to reduce the severity or symptoms of cancer, such as pain relief, tumor volume shrinkage, or reduction of metastasis of tumor cells. The tumor volume can be determined clinically by palpable mass or by various medical imaging means.

As used herein, the term “overexpression” means that in a cell or an organism, the expression level of a gene product exceeds that in a normal cell or a normal organism.

According to some embodiments of the present disclosure, the use of bupropion in the preparation of drugs for cancer treatment is provided, in which the tumor cells of the cancer overexpress CHRNA9.

According to some embodiments of the present disclosure, a pharmaceutical composition containing an effective amount of bupropion and an excipient is provided for the preparation of drugs for cancer treatment, in which the tumor cells of the cancer overexpress CHRNA9.

According to some embodiments of the present disclosure, a method of inhibiting migration of tumor cells is provided, wherein the tumor cells overexpress CHRNA9. The method includes: administrating an effective amount of bupropion to the tumor cells.

According to some embodiments of the present disclosure, a method for treating cancer is provided. The method includes administering an effective amount of bupropion to a subject having cancer, wherein the tumor cells of the cancer overexpress CHRNA9.

According to some embodiments of the present disclosure, a method for treating cancer is provided. The method includes administering a pharmaceutical composition to a subject having cancer. The pharmaceutical composition includes an effective amount of bupropion and an excipient.

According to some embodiments of the present disclosure, the use of bupropion is provided for treating cancer, in which the tumor cells of the cancer overexpress CHRNA9.

In some embodiments, the disclosure provides a use of bupropion for cancer treatment, wherein nicotine or nicotine derivative is an inducing factor of this cancer.

In some embodiments, bupropion reduces metastasis of tumor cells.

In some embodiments, the present disclosure provides a use of bupropion for treating cancer, wherein the cancer is breast cancer, head and neck squamous cell carcinoma, lung adenocarcinoma, or uterine corpus endometrial carcinoma.

In some embodiments, the present disclosure provides a use of bupropion for treating cancer, wherein the cancer is triple-negative breast cancer.

According to some embodiments, the pharmaceutical composition containing bupropion is an oral formulation.

In some embodiments, the disclosure provides a method of inhibiting migration of tumor cells, wherein the tumor cells are triple-negative breast cancer (TNBC) cells.

In some embodiments, the disclosure provides a method of inhibiting migration of tumor cells, wherein the method is carried out in vitro.

Previous studies by the inventors have shown that nicotine or nicotine derivatives activated CHRNA9, and then made breast cell cancerous and caused tumor formation. But so far, Galantamine, a drug for dementia, is the only one drug targeting CHRNA9. There is no drug for cancer treatment in which the target protein is CHRNA9.

In some embodiments, the formulation of bupropion is oral. The oral formulation of the pharmaceutical compositions disclosed in the present disclosure may be a formulation of tablet, half tablet, capsule, or liquid (e.g., syrup). These formulations contain a predetermined amount of active ingredients and can be manufactured by methods known in the art.

The typical oral formulation is prepared by mixing the active ingredient with at least one excipient in accordance with conventional pharmaceutical methods. The choice of excipients depends on the types of preparation.

Oral administration is an easier way for administration, and the most common type of oral formulation is tablet and capsule. If necessary, a standard solution or non-solution coating method can be used to coat the outside of the tablet or capsule. In general, the formulation is manufactured by evenly mixing the active ingredient with a liquid carrier, a solid carrier, or both, and then molding into a suitable shape. A disintegrating agent can be added to help the drug dissolve quickly, or a lubricant can be added to help the manufacture process of the drug.

According to some embodiments, the formulation of bupropion may be immediate release form, sustained release form, or extended release form.

According to some embodiments, bupropion is administered from one to four times a day. For example, the immediate release form is administered three or four times a day; the sustained release form is administered twice a day; the extended release form is administered once a day.

One of the main known side effects of bupropion is the incidence of epilepsy, which is known to be positively correlated with the dose of bupropion. In some embodiments, the patient is administered no more than 600 mg of bupropion per day.

In some embodiments, the amount of bupropion the pharmaceutical composition is from about 25 mg to about 600 mg, such as 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, or 600 mg.

In order to identify cancer types overexpressing CHRNA9, the expressions of Chrna9 gene in 15 cancers in the American Cancer Genome Atlas (TCGA) database were analyzed, as shown in Table 1 below. In some cancer types, such as breast cancer, head and neck squamous cell carcinoma, lung adenocarcinoma, or uterine corpus endometrial carcinoma, the average expression of CHRNA9 in all tissues was twice or more than that in all normal tissues, and the P-values were less than 0.05. This indicates that CHRNA9 expression was significantly higher in these cancer types. Therefore, in these cancer types, CHRNA9 may be used as a target of drug therapy.

TABLE 1
The gene expression of CHRNA9 in 15 cancer types in TCGA
database.

| | Abbreviated name | | |
|---|---|---|---|
| | (No. of | | |
| | normal samples/ | Fold | |
| | No. of | change | |
| Cancer types | tumor samples) | (log₂) | P-value |
| Bladder urothelial carcinoma | BLCA (19/408) | 0.48 | 0.209 |
| Breast invasive carcinoma | BRCA (113/1102) | 1.18 | 0.000 |
| Cholangiocarcinoma | CHOL (9/36) | 0.15 | 0.324 |
| Colon adenocarcinoma | COAD (41/287) | 0.11 | 0.159 |
| Head and neck squamous cell | HNSC (44/522) | 1.08 | 0.001 |
| carcinoma | | | |
| Kidney chromophobe | KICH (25/66) | 0.22 | 0.110 |
| Kidney renal clear cell | KIRC (72/534) | 0.00 | 0.973 |
| carcinoma | | | |
| Kidney renal papillary cell | KIRP (32/291) | 0.13 | 0.160 |
| carcinoma | | | |
| Liver hepatocellular carcinoma | LIHC (50/374) | 0.18 | 0.087 |
| Lung adenocarcinoma | LUAD (59/517) | 1.32 | 0.000 |
| Lung squamous cell carcinoma | LUSC (51/502) | 0.46 | 0.062 |
| Prostate adenocarcinoma | PRAD (52/498) | −0.06 | 0.380 |
| Rectum adenocarcinoma | READ (10/95) | −0.10 | 0.620 |
| Thyroid carcinoma | THCA (59/513) | −0.29 | 0.111 |
| Uterine corpus endometrial | UCEC (24/177) | 1.17 | 0.006 |
| carcinoma | | | |

Bold letters represent that CHRNA9 is significant up-regulation in these cancer types (The change of gene expression was greater than two times, and the P-value was less than 0.05).

The following experimental examples in the present disclosure are based on breast cancer cell lines and model animals for testing CHRNA9 as a target of cancer drug therapy.

Neural acetylcholine receptor (nAChR) is a membrane receptor of a neurotransmitter and an ion channel. Among nAChRs, CHRNA9 is known to be associated with a variety of smoking-induced tumor formation. For example, the inventors' previous studies showed that CHRNA9 highly expressed (mean 7.84 fold) in 186 (67.3%) of 276 breast cancer paired samples. However, the interacting partners and pathways associated with CHRNA9 remain to be elucidated.

Using bioinformation analysis methods, the inventors initially identified 18 candidate proteins which may interact with CHRNA9. The candidate proteins are ERBB3, ERBB2, SFN, COPSE, SRC, CSNKID, ERBB4, INSR, ATXN1, ABCB1, APP, YWHAB, YWHAG, EGFR, HCK, FYN, PLK1, and YWHAH.

The following experimental examples illustrate the mechanisms related to CHRNA9 and the application of CHRNA9 as a target of drug therapy.

In the experimental examples of the present disclosure, the data were analyzed by Student's t-test. All P-values were derived from two-tail test. If a p-value is less than 0.05, it is flagged with one star (*); if a p-value is less than 0.01, it is flagged with two stars (**).

### EXAMPLE 1

To determine whether these 18 candidate proteins interact with CHRNA9 in human breast cancer cells, expressions of CHRNA9 and other relevant proteins in different breast cancer lines were characterized via immunoprecipitation (IP) assay and Western Blot (WB).

Triple-negative breast cancer (TNBC) is a breast cancer subtype with negative expressions of estrogen receptor (ER) and progesterone receptor (PR), and no excessive expression of human epidermal growth factor receptor 2 (HER2, also called ERBB2).

FIG. 1A shows the Western Blot image. The selected cell lines are two breast normal (non malignant) cell lines (MCF-10A and HBL-100) and six breast cancer cell lines including luminal subtype (MCF-7 and T47D), HER2-enriched subtype (MDA-MB-453 and SKBR3) and TNBC subtype (MDA-MB-157 and MDA-MB-231). FIG. 1A shows the expressions of CHRNA9, 5 candidate proteins (EGFR, ERBB2, ERBB3, FYN, and SRC), ER, and PR in different breast cancer cell lines. GAPDH antibody was used for positive control.

FIG. 1B shows a Western Blot image of the immunoprecipitation (IP) samples. Accordingly, proteins interacting with CHRNA9 can be identified. During the IP assay, the protein lysates of MDA-MB-231 cells were mixed with anti-CHRNA9-IgG magnetic beads for precipitation. Then Western blotting was carried out to detect proteins in the pellet, and GAPDH antibody was used for negative control. FIG. 1B shows that the 16 of the 18 candidate proteins, COPSE, CSNK1D, FYN, ERBB2, ERBB3, SFN, SRC, ERBB4, INSR, ATXN1, ABCB1, APP, YWHAB, YWHAG, EGFR, and HCK, bound to CNRNA9. Therefore, these proteins may interact with CHRNA9 and form a complex.

In order to investigate the interaction relationship between CHRNA9 with YWHAG or SNF, CHRAN9 in MDA-MB-231 cells were labeled with a Rhodamine conjugated antibody (red fluorescence), and YWHAG and SFN were respectively labeled with a Fluorescein isothiocyanate (FITC) conjugated antibody (green fluorescence). In the merged fluorescence images, yellow color indicates the colocalization of the two fluorescent-labeled proteins. Further, Förster resonance energy transfer (FRET) was used to observe the strength of the interaction between CHRNA9 with YWHAG or SFN. When the color of FRET is closer to yellow-green, the stronger the interaction strength between the two proteins; conversely, when the color is closer to blue-black, the weaker the interaction strength between the two proteins, even no interaction between the two proteins.

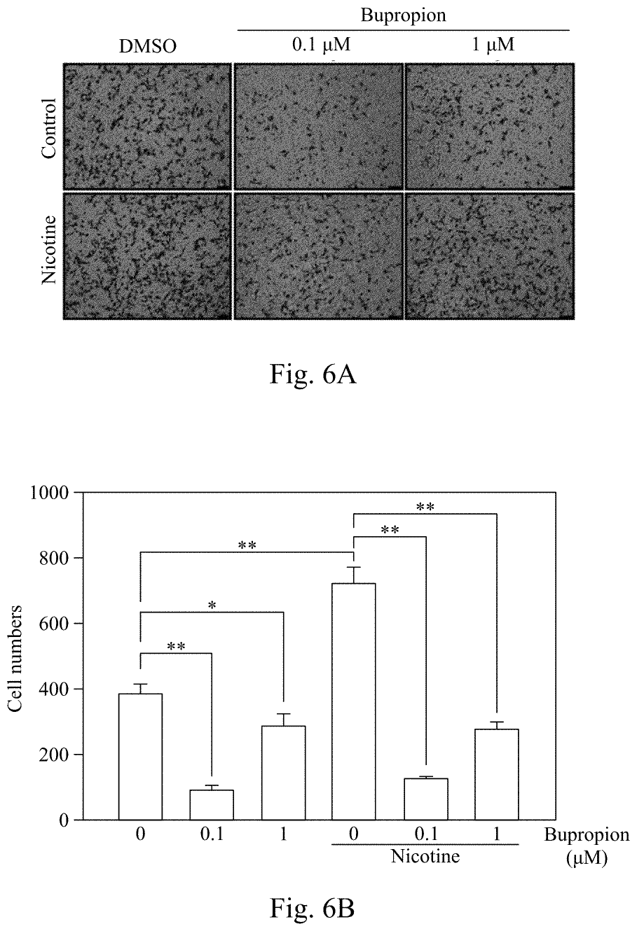

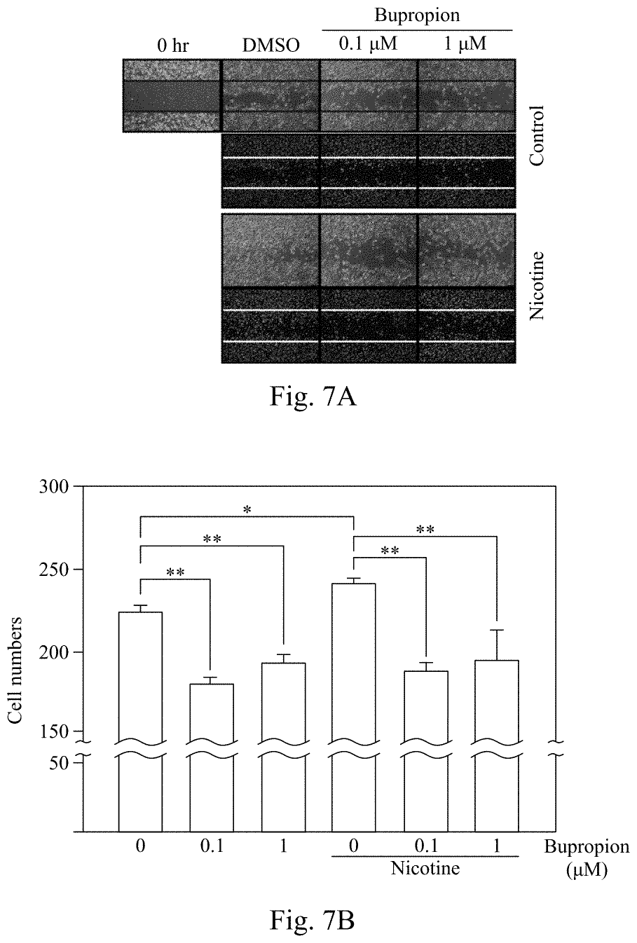

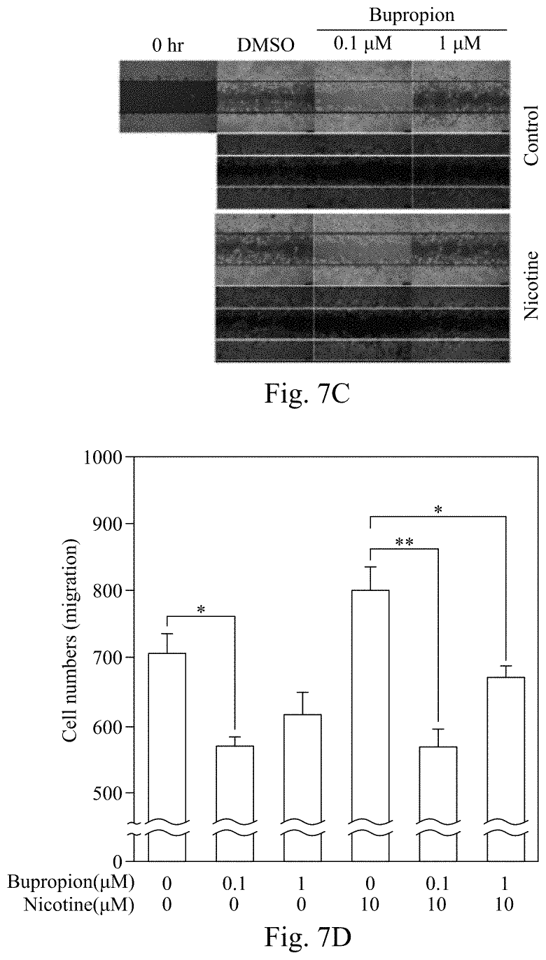

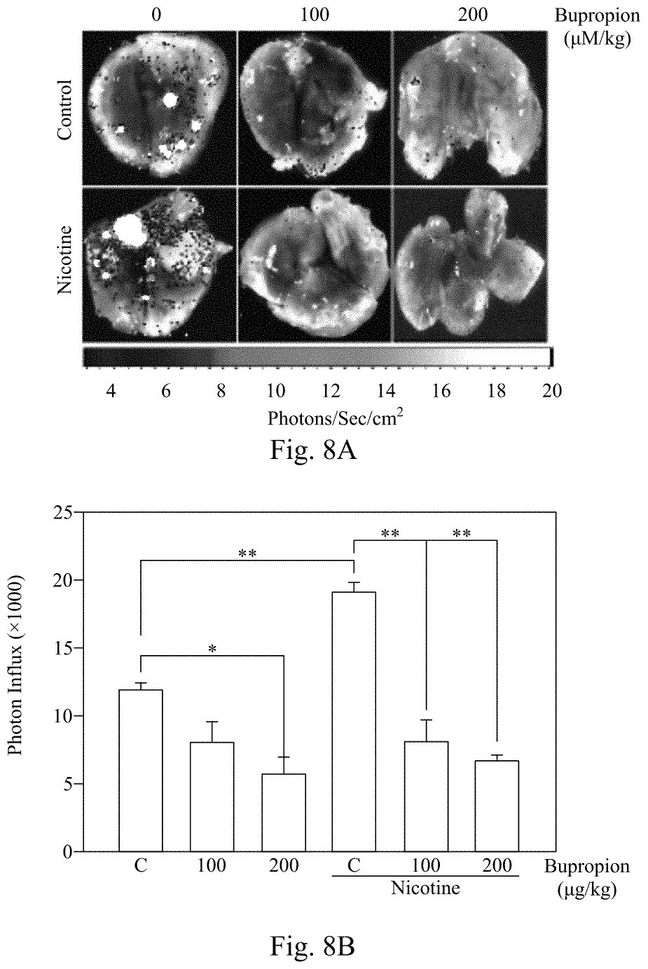

As shown in FIG. 10, the upper and lower rows are two series of fluorescence images of MDA-MB-231 cells. The green fluorescence (light color) in the cells indicates the expression of YWHAG, the red fluorescence (light color) in the cells indicates the expression of CHRNA9, and the yellow color (light color) in the merged image indicates the colocalization of YWHAG and CHRNA9. The yellow-green (light color) FRET activity in FRET images indicates that CHRNA9 and YWHAG were very near to one another, so the two proteins interacted with each other.

As shown in FIG. 1 D, the upper and lower rows are two series of fluorescence images of MDA-MB-231 cells. The green fluorescence (light color) in the cells indicates the expression of SFN, the red fluorescence (light color) in the cells indicates the expression of CHRNA9, and the yellow color (light color) in the merged image indicates the colocalization of SFN and CHRNA9. The yellow-green (light color) FRET activity in FRET images indicates that CHRNA9 and SFN were very near to one another, so the two proteins interacted with each other.

### EXAMPLE 2

To more confirm whether CHRNA9 interacts with ERBB2, further examinations were carried out on HER2 overexpression cell lines (SKBR3, BT474, AU565, HCC1419, and HCC1954) and on a TNBC cell line (MDA-MB-468). FIG. 2A shows the result of Western blotting, the expressions of CHRNA9 and HER2 (including p185, HER2, and p95) in different subtypes of breast cancer cell lines, and a-Tubulin was used for positive control.

In order to further investigate the interaction between CHRNA9 and ERBB2 ex vitro, fluorescent conjugated antibodies were used, and CHRNA9 was stained with Rhodamine (red fluorescence), ERBB2 was stained with FITC (green fluorescence) in BT474 cell line and in cancer cells isolated from clinical samples (three samples were HER2 overexpression subtype and one sample was TNBC subtype). FRET was also used to observe the interaction strength between CHRNA9 and ERBB2. When the color of FRET is closer to yellow-green, the stronger of the interaction strength between the two proteins; conversely, when the color of FRET is closer to blue-black, the weaker the interaction strength between the two proteins, even no interaction between the two proteins.

As shown in FIG. 2B, the upper and lower rows are two series of fluorescence images of BT474 cells. The green fluorescence (light color) in the cells indicates the expression of HER2 (ERBB2), the red fluorescence (light color) in the cells indicates the expression of CHRNA9, and the yellow color (light color) in the merged image indicates the colocalization of HER2 and CHRNA9. The yellow-green (light color) FRET activity in FRET images indicates that CHRNA9 and HER2 were very near to one another, so the two proteins interacted with each other.

FIG. 2C shows the tissues from clinical samples of breast cancer patients, in which three samples were HER2 overexpression subtype and one sample was TNBC subtype. In the tissues of HER2 overexpression subtypes, green fluorescence (light color) indicates HER2 (ERBB2) expression; while no green fluorescence was present in the tissue of TNBC subtype. The red fluorescence indicates the expression of CHRNA9, and the yellow color (light color) in the merged image indicates the colocalization of HER2 and CHRNA9. The yellow-green (light color) FRET activity in FRET images indicates that CHRNA9 and HER2 were very near to one another, so the two proteins interacted with each other.

### EXAMPLE 3

In order to further investigate the interaction between CHRNA9 and ERBB2 ex vivo, a plasmid expressing CHRNA9/CFP and a plasmid expressing ERBB2/YFP were constructed and then co-transfected into MDA-MB-231 cells. FRET and fluorescence-lifetime imaging microscopy (FLIM) were used.

To further clarify whether CHRNA9 and ERBB2 are dissociated after nicotine exposure, two-photon FLIM was used to monitor the FRET activity between CHRNA9/CFP and ERBB2/YFP. The plasmids expressing CHRNA9/CFP and the plasmids expressing ERBB2/YFP were co-transfected into MDA-MB-231 cells. After two days, the cells were treated with 10 μM nicotine.

FIG. 3 shows that before the treatment of 10 μM nicotine (pre-treatment), the FRET activity on the membranes was strong (green, light color), while the FRET activity in the plasma of the cells was weak (blue, dark color). After nicotine exposure, the fluorescence on the cell membrane gradually turned to blue; this indicates the FRET activity gradually decreased.

Then the cells were washed with PBS to remove nicotine, and the fluorescence on the membrane gradually turned to green; this indicates the FRET activity gradually recovered to the baseline (before nicotine treatment).

### EXAMPLE 4

Verifying nicotine can result in the dissociation between CHRNA9 and ERBB2 in an animal model.

Split luciferase complementation assay was used to investigate the association and dissociation between CHRNA9 and ERBB2.

FIG. 4A is a schematic diagram of the split luciferase complementation assay. The N terminal (Nluc) and the C terminal (Cluc) of luciferase were respectively constructed with ERBB2 and CHRNA9 to form the plasmids expressing the fusion proteins. When ERBB2 and CHRNA9 interact with each other, the Nluc and the Cluc are also close to each other to assemble together, and present luciferase activity. Therefore, after Luciferin addition, luminescence can be generated. When ERBB2 and CHRNA9 dissociate from each other, the Nluc and Cluc also dissociate from each other; therefore, no luciferase activity exists. Some experimental examples in the present disclosure related to testing the luciferase activity after nicotine addition. If no luminescence is generated after Luciferin addition, this means nicotine cause ERBB2 and CHRNA9 dissociate from each other.

FIG. 4B shows the results of Western blotting from cells co-transfected with plasmids expressing the fusion proteins. Anti-CHRNA9 and anti-ERBB2 antibodies were used to detect the expression levels of the CHRNA9 fusion protein and the ERBB2 fusion protein. FIG. 4B shows that the expression levels of the CHRNA9/Cluc fusion protein and the ERBB2/Nluc fusion protein correlated with the doses of the plasmids co-transfected into the cells.

Then a xenograft animal model was used. Plasmids expressing ERBB2/Nluc fusion protein and plasmids expressing CHRNA9/Cluc fusion protein were co-transfected into MDA-MB-231 cells, then the cells expressing the fusion proteins were injected into the mammary pads of nude mice. Then the mice were exposed to nicotine by oral administration. IVIS was used to measure the luminescent signals before nicotine exposure (control) and at 30 minutes of nicotine exposure. As shown in FIG. 4C, the image of the control group shows the tumor cells expressed a higher level of luciferase activity in the tumor region. After the mice were exposed to nicotine, the luciferase activity in the tumor region significantly reduced. In nicotine treatment group, the photon influx was 26.3% of that of the control group. This indicates that in this animal model, nicotine significantly caused dissociation of the CHRNA9/ERBB2 complex.

### EXAMPLE 5

The above results can be inferred that a drug capable of preventing the dissociation between CHRNA9 and ERBB2 may affect the downstream signaling pathways of tumor cells. Therefore, 1,543 FDA-approved drugs were then screened to identify drugs that could prevent dissociation between CHRNA9 and ERBB2. In addition, based on protein-drug interaction profiles and conservation binding environments mined from 33 protein-ligand nACHR structures, bupropion was identified as it may prevent dissociation between CHRNA9 and ERBB2.

FIG. 5A illustrates the molecular structure of nicotine. FIG. 5B shows the molecular structure of bupropion. FIG. 5C shows a partial protein structure of CHRNA9 to illustrate the relative spatial locations of bupropion, nicotine, and the nearby amino acid residues of CHRNA9. FIG. 5C shows that bupropion docked into an allosteric binding site, and the 1-(3-chlorophenyl) propan-1-one of bupropion formed strong van der Waals forces with the contact residues I140, G141, S142 and D194.

### EXAMPLE 6

Tumor metastasis is an important factor in determining the survival of cancer patients. Therefore, the following assay is to test whether bupropion can be a blockade to attenuate nicotine-induced cancer metastatic effects.

The inhibitory effect of bupropion on tumor invasion was tested by cell invasion assay. MDA-MB-231 cells were plated on to Matrigel invasion chambers in serum-starved medium, and the cells in the lower chamber were cultured using normal medium. The media in both of the upper and the lower chambers contained 0.1 μM bupropion, or 1 μM bupropion, and the cells were treated with or without (control group) nicotine. After the cells were cultured for 48 hours, the upper chambers were washed with PBS and fixed with formaldehyde for 30 minutes. The cells in the upper chambers were stained with crystal violet for two hours, and photos were acquired under a microscope. The numbers of invading tumor cells were calculated by ImageJ software.

FIG. 6A shows an image of the upper chamber after crystal violet staining. FIG. 6B is a bar graph based on the quantification of the cells in FIG. 6A. FIGS. 6A and 6B show that the bupropion treatment at 0.1 μM and 1 μM significantly inhibited the invasion of MDA-MB-231 cells. The bupropion treatment at lower concentration (0.1 μM) had a more significant effect.

### EXAMPLE 7

Cell migration assay was used to test the inhibitory effect of bupropion on tumor cell migration.

MDA-MB-231 cells well treated with or without (control group) nicotine, DMSO, 0.1 μM, and 1 μM bupropion were then respectively added to the cells. Twelve hours after bupropion addition, the cells were fixed with formaldehyde, and stained with PI for 30 minutes; then photos of the cells were acquired. The numbers of migrating cells were calculated by imageJ software. This assay was repeated 3 times.

FIG. 7A shows the images of the cells, and FIG. 7B is a bar graph based on the quantification of the cells in FIG. 7A. As shown in FIGS. 7A and 7B, the bupropion treatment at 0.1 μM and 1 μM significantly inhibited the migration of MDA-MB-231 cells.

### EXAMPLE 8

In addition, a HER2 overexpression subtype of a breast cancer cell line, MDA-MB-453, was used to test the inhibitory effect of bupropion on tumor migration. The experimental method was the same as the cell migration assay of Example 7 above.

FIG. 7C shows the images of the cells, and FIG. 7D shows the bar graph based on the quantification of the cells in FIG. 7C. FIGS. 7C and 7D show that the bupropion treatment at 0.1 μM and 1 μM significantly inhibited the migration of MDA-MB-453 cells.

### EXAMPLE 9

Testing bupropion as a blockade of nicotine in an MDA-MB-231-based spontaneous pulmonary metastasis animal model.

MDA-MB-231 cells with a luminescence reporter gene were implanted into severe combined immunodeficiency (SCID) mice. The nicotine treatment was carried out by adding nicotine (10 μg/ml) to the drinking water of the mice. The mice were injected intraperitoneally with bupropion three times a week at doses of 100 or 200 μg per kilogram. After two months, the mice were sacrificed and the lungs were dissected. IVIS was used to take the luminescence images of the lungs. The photon influx measured in the images represents the metastasis level of the mouse tumor cells to the lung tissue.

FIG. 8A shows the luminescence images of the lungs of the mice; FIG. 8B shows the bar graph based on the quantification of the photon flux in FIG. 8A.

FIGS. 8A and 8B show that in mice treated with nicotine, the number of the metastatic tumor cells in the lung tissues significantly increased. In addition, in both nicotine-free and nicotine-treated mice received bupropion, the number of the migrating tumor cells to the lungs significantly decreased. This suggests that bupropion blocked signals from nicotine and may also inhibit signals from other endogenous nACHRs agonists.

TNBC accounts for about 15-20% of all breast cancers, and it is more difficult to be treated than other subtypes. According to clinical statistics, TNBC has high probabilities of metastasis and recurrence. For example, the recurrence rate of TNBC has a peak of about one to three years after treatment, especially for young women (before 40 years of age). The process of cancer progression to metastasis in TNBC is quick, at its mortality is also high. At present, there is no effective targeted therapy for TNBC, so TNBC treatment mainly relies on chemotherapy and radiotherapy. The available treatment options are significantly less than other subtypes of breast cancer; therefore, TNBC patients usually have a poor prognosis. Therefore, it has an emerging need for discovering and developing effective targeted drugs to reduce TNBC's recurrence and metastasis rates.

Cell experiments and animal experiments of the present disclosure demonstrate that the migration or the metastasis ability of TNBC cells (such as MDA-MB-231) and HER2 overexpression subtype breast cancer cells (MDA-MB-453) were significantly decreased after bupropion addition. Also, bupropion inhibited the nicotine-induced tumor migration and metastasis. Therefore, bupropion can be an anti-metastatic drug for breast cancer, such as TNBC or HER2 overexpression subtypes of breast cancer.

The experimental examples in the present disclosure show that bupropion affected gene expression of metastasis-related signaling pathways and attenuated nicotine-induced cell metastasis. Therefore, bupropion can be an anticancer drug; in particular, anti-metastasis drug.

Because bupropion is a known compound for clinical use, the safety and side effects of bupropion are well understood. Accordingly, the present disclosure provides a more convenient, effective, and lower side-effect treatment option for cancer therapy.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

1.-13. (canceled)

14. A method for inhibiting migration of tumor cells, the method comprising:
administrating an effective amount of bupropion to the tumor cells;
wherein the tumor cells overexpress CHRNA9.

15. The method according to claim 14, wherein the tumor cells are breast cancer cells.

16. The method according to claim 14, wherein the tumor cells are triple-negative breast cancer cells.

17. The method according to claim 14, wherein the method is carried out in vitro.
